(19) United States
(12) Patent Application Publication
(10) Pub. No.: US 2025/0041055 A1
(43) Pub. Date: Feb. 6, 2025

(54) PRESSURE-REDUCTION SYSTEMS AND METHODS FOR PROSTHETIC VALVES

(71) Applicant: EDWARDS LIFESCIENCES CORPORATION, Irvine, CA (US)

(72) Inventor: Nadav Ben Zaken, Netanya (IL)

(21) Appl. No.: 18/924,330

(22) Filed: Oct. 23, 2024

Related U.S. Application Data
(63) Continuation of application No. PCT/US2023/020469, filed on Apr. 28, 2023.
(60) Provisional application No. 63/364,404, filed on May 9, 2022.

(51) Int. Cl. A61F 2/24

(52) U.S. Cl. CPC A61F 2/2418; A61F 2/243; A61F 2220/0025

(57) ABSTRACT

A prosthetic valve assembly comprises a docking structure having an inflow end and an outflow end, the docking structure having a lumen extending between the inflow end and the outflow end; and a prosthetic heart valve disposed within the lumen of the docking structure, the prosthetic heart valve comprising a frame and an occluding structure disposed within the frame, the occluding structure configured to regulate a flow of blood through the frame in one direction; wherein the docking structure is configured to allow the prosthetic heart valve to be movable relative to the docking structure in an axial direction between the inflow end and the outflow end of the docking structure during flow cycles of a heart.

### CROSS-REFERENCE TO RELATED APPLICATIONS

This application is a continuation of PCT patent application number PCT/US2023/020469 filed on Apr. 28, 2023, which claims the benefit of U.S. Provisional Application No. 63/364,404, filed May 9, 2022, the entireties of both of these applications being incorporated by this reference herein.

### FIELD

The present disclosure relates to prosthetic valves and systems and methods for prosthetic valves that are moveable relative to a docking structure for pressure reduction purposes.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable.

In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

### SUMMARY

Described herein are prosthetic valve assemblies, delivery apparatuses, and methods for implanting prosthetic valve assemblies. The disclosed prosthetic valve assemblies and methods can, for example, reduce undesirable pressure gradients across prosthetic heart valves. As such, the devices and methods disclosed herein can, among other things, overcome one or more of the deficiencies of typical prosthetic valves.

A prosthetic valve assembly can comprise a prosthetic heart valve and a docking device. In addition to these components, a prosthetic valve assembly can further comprise one or more of the components disclosed herein.

In some examples, a prosthetic valve assembly can comprise a prosthetic heart valve that is movable relative to a docking structure in an axial direction between an inflow end and an outflow end of the docking structure during flow cycles of a heart.

In some examples, a prosthetic valve assembly can comprise a docking device having first and second stops at axially spaced locations.

In some examples, a prosthetic valve assembly can comprise a prosthetic heart valve and a docking device, wherein the prosthetic valve assembly is configured to transition between a forward flow configuration and a reverse flow configuration in response to flow cycles of a heart, wherein the forward flow configuration comprises a first blood flow pathway extending through the prosthetic heart valve and a second blood flow pathway extending through an opening between an outer surface of the prosthetic heart valve and an inner surface of the docking device.

In some examples, a prosthetic heart valve comprises one or more of the components recited in Examples 1-62 below.

In one representative example, a prosthetic valve assembly comprises a docking structure having an inflow end and an outflow end, the docking structure having a lumen extending between the inflow end and the outflow end; and a prosthetic heart valve disposed within the lumen of the docking structure, the prosthetic heart valve comprising a frame and an occluding structure disposed within the frame, the occluding structure configured to regulate a flow of blood through the frame in one direction; wherein the docking structure is configured to allow the prosthetic heart valve to be movable relative to the docking structure in an axial direction between the inflow end and the outflow end of the docking structure during flow cycles of a heart.

In another representative example, a docking device comprises a base having an inflow end and an outflow end, the base comprising a lumen extending from the inflow end to the outflow end, wherein the docking device is configured to receive a prosthetic heart valve such that the prosthetic heart valve is axially moveable within the lumen between the inflow end and the outflow end of the docking device during flow cycles of a heart.

In another representative example, a prosthetic valve assembly comprises a docking device having an inflow end and an outflow end; and a prosthetic heart valve positioned within the docking device between the inflow end and the outflow end; wherein the prosthetic valve assembly is configured to transition between a forward flow configuration and a reverse flow configuration in response to flow cycles of a heart, wherein the forward flow configuration comprises a first blood flow pathway extending through the prosthetic heart valve from the inflow end to the outflow end and a second blood flow pathway extending from the inflow end to the outflow end between an outer surface of the prosthetic heart valve and an inner surface of the docking device.

In another representative example, a method of delivering a prosthetic valve assembly to an implantation location within a patient comprises advancing a docking device retained on a first delivery device to an implantation location within a patient; radially expanding the docking device at the implantation location; advancing a prosthetic heart valve retained on a second delivery device to the implantation location; positioning the prosthetic heart valve between inflow and outflow ends of the docking device; and radially expanding the prosthetic heart valve within the docking device.

In another representative example, a method of delivering a prosthetic valve assembly to an implantation location within a patient comprises advancing a docking device retained on a first expansion mechanism of a delivery device and a prosthetic heart valve retained on a second expansion mechanism of the delivery device to an implantation location within a patient; radially expanding, by the first expansion mechanism, the docking device at the implantation location; positioning the prosthetic heart valve between inflow and outflow ends of the docking device; and radially expanding, by the second expansion mechanism, the prosthetic heart valve within the docking device.

In another representative example, a method of delivering a prosthetic valve assembly to an implantation location within a patient comprises advancing a prosthetic valve assembly retained within a sheath of a delivery device to an implant location within a patient, the prosthetic valve assembly comprising a docking structure and a prosthetic heart valve positioned between an inflow end and an outflow end of the docking structure; deploying the prosthetic valve assembly from the sheath to allow the docking structure to self-expand at the implantation location; and radially expanding, by an expansion mechanism of the delivery device, the prosthetic heart valve within the docking device.

In another representative example, a prosthetic valve assembly comprises a docking device having an inflow end and an outflow end and first and second stops at axially spaced locations; and a prosthetic heart valve configured to be positioned within the docking device between the first and second stops; wherein the docking device is configured to allow the prosthetic heart valve to move axially within the docking device in first and second directions between the first and second stops during flow cycles of a heart.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side elevation view of a prosthetic valve, according to one example.

FIG. 2 is a side view of an example of a delivery apparatus configured to deliver and implant a radially expandable medical device at an implantation site.

FIG. 3 is a side elevation view of a prosthetic valve assembly that includes the prosthetic valve of FIG. 1 positioned within a docking device (shown in cross-section) under the forward flow of blood, according to one example.

FIG. 4 is a side elevation view of the prosthetic valve assembly of FIG. 3 under the reverse flow of blood.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

### Overview of the Disclosed Technology

Prosthetic valve assemblies disclosed herein can include configurations that aid in alleviating pressure and/or reducing afterload during systolic-diastolic flow cycles of a heart. In particular, the prosthetic valve assemblies can transition between a forward flow configuration and a reverse flow configuration during flow cycles to alleviate pressure and reduce afterload. For example, the prosthetic valve assemblies disclosed herein can include a docking device and a prosthetic heart valve that is moveable relative to the docking device when the prosthetic heart valve is implanted within a lumen of the docking device. As the prosthetic heart valve moves or transitions to the forward flow configuration, multiple blood flow pathways are defined through the prosthetic valve assembly (e.g., a pathway through the prosthetic heart valve, a pathway between the prosthetic heart valve and the docking device, etc.). As will be described in more detail below, the multiple pathways allow competitive flow through the prosthetic valve assembly to reduce pressure at the implant location (e.g., pressure across the aortic annulus, etc.). In some examples, this pressure reduction can also reduce afterload (e.g., pulsatile LV afterload, etc.) which can be beneficial for patients, including those with hypertensive heart failure.

In some examples, the docking device can include stoppers (e.g., positioned at inflow and outflow ends of the docking device, etc.) that retain or hold the prosthetic heart valve within the docking device, while still allowing movement of the prosthetic heart valve in an axial or longitudinal direction relative to the docking device. In particular, the prosthetic heart valve can move axially between the stoppers during flow cycles of the heart, without moving past or beyond the stoppers. Examples of prosthetic heart valves, delivery apparatuses, docking devices, and associated methods of use will be discussed in more detail below.

### Examples of the Disclosed Technology

The prosthetic valve assemblies disclosed herein are primarily intended for implantation within the native aortic valve for treating patients with hypertensive heart failure. However, in other examples they can be adapted to be implanted in the other native annuluses of the heart (the pulmonary, mitral, and tricuspid valves). The disclosed prosthetic valve assemblies also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valve assemblies also can be implanted within a previously implanted prosthetic valve (which can be a prosthetic surgical valve or a prosthetic transcatheter heart valve) in a valve-in-valve procedure.

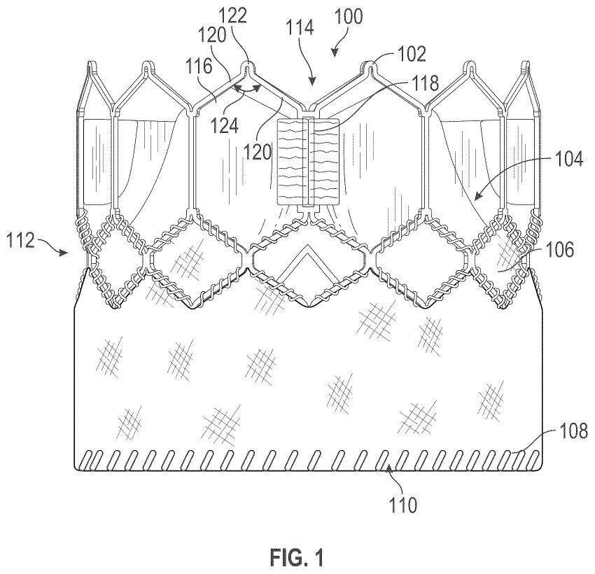

FIG. 1 shows an exemplary prosthetic valve 100 (which can be referred to as a “prosthetic heart valve”) that can be used in combination with docking devices disclosed herein to form a prosthetic valve assembly. The prosthetic valve 100 comprises four main components: a stent or frame 102, an occluding structure 104 (e.g., a valvular structure, etc.), an inner skirt 106, and an outer sealing member or outer skirt 108. The prosthetic valve 100 can have an inflow end portion 110, an intermediate portion 112, and an outflow end portion 114. The inner skirt 106 can be arranged on and/or coupled to an inner surface of the frame 102, while the outer skirt 108 can be arranged on and/or coupled to an outer surface of the frame 102.

The occluding structure 104 can comprise three leaflets 116, collectively forming a leaflet structure, which can be arranged to collapse in a tricuspid arrangement, although in other examples there can be greater or fewer number of leaflets (e.g., one or more leaflets 116). The leaflets 116 can be secured to one another at their adjacent sides to form commissures 118 of the occluding structure 104. The lower edge of occluding structure 104 can have an undulating, curved scalloped shape and can be secured to the inner skirt 106 by sutures (not shown). In some examples, the leaflets 116 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein. In some examples, the occluding structure 104 can comprise other types of members (e.g., members other than leaflets 116) to regulate blood flow, such as a flap, a duck-bill type valve, etc.

The frame 102 can be radially compressible (collapsible) and expandable (e.g., expanded configuration shown in FIG. 1) and comprise a plurality of interconnected struts 120. A plurality of apices 122 that are spaced circumferentially apart are formed at the inflow end portion 110 and the outflow end portion 114 of the frame 102 (only the apices 122 at the outflow end portion 114 are visible in FIG. 1). Each apex 122 is formed at a junction between two angled struts 120 at either the inflow end portion 110 or the outflow end portion 114. FIG. 1 depicts a known frame design with apices 122 that form a U-shaped bend between the two angled struts 120. In some examples, an angle 124 between the two angled struts 120, connected at the apex 122, can be in a range of 90 to 120 degrees.

The frame 102 can be formed with a plurality of circumferentially spaced slots, or commissure windows that are adapted to mount the commissures 118 of the occluding structure 104 to the frame. The frame 102 can be made of any of various suitable plastically expandable materials (e.g., stainless steel, etc.) or self-expanding materials (e.g., Nitinol). When constructed of a plastically expandable material, the frame 102 (and thus the prosthetic valve 100) can be crimped to a radially collapsed configuration on a delivery catheter or apparatus and then expanded inside a patient by an inflatable balloon or equivalent expansion mechanism. When constructed of a self-expandable material, the frame 102 (and thus the prosthetic valve 100) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frame 102 include metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35NTM/UNS R30035comprises 35% nickel, 35% cobalt, 20% chromium, and 10% molybdenum, by weight. Additional details regarding the prosthetic valve 10 and its various components are described in WIPO Patent Application Publication No. WO 2018/222799, which is incorporated herein by reference.

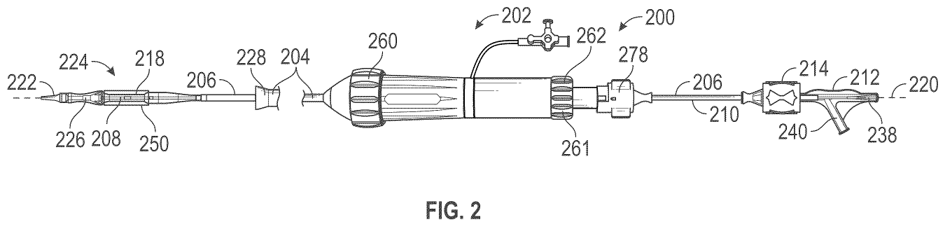

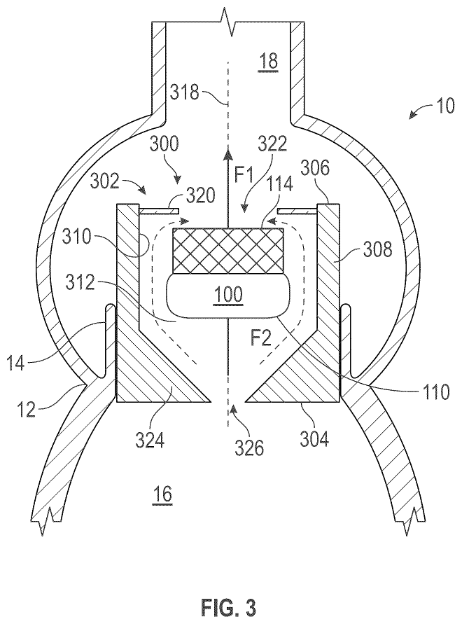

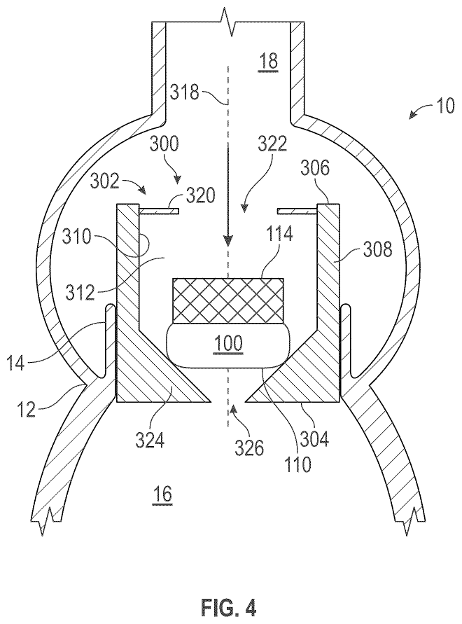

FIG. 2 shows a delivery apparatus 200, according to an example, that can be used to implant an expandable medical device, such as an expandable prosthetic heart valve (e.g., the prosthetic heart valve 100 of FIG. 1 and/or any of the other prosthetic heart valves described herein) or an expandable docking device (e.g., the docking device 302 of FIGS. 3-4 and/or any of the other docking devices described herein). In some examples, the delivery apparatus 200 is specifically adapted for use in introducing a medical device into a heart.

The delivery apparatus 200 in the illustrated example of FIG. 2 is a balloon catheter comprising a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (e.g., move) longitudinally, along a central longitudinal axis 220 of the delivery apparatus 200, relative to one another to facilitate delivery and positioning of a prosthetic valve at an implantation site in a patient's body.

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 204.

The adaptor 212 can include a first port 238 configured to receive a guidewire therethrough and a second port 240 configured to receive fluid (e.g., inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an expansion mechanism 218 of the delivery apparatus 200. A distal end portion of the inner shaft 208 can extend distally beyond the distal end portion of the intermediate shaft 206.

In the illustrated example, the expansion mechanism 218 comprises an inflatable balloon. The balloon 218 can be coupled to the distal end portion of the intermediate shaft 206. In some examples, a distal end of the balloon 218 can be coupled to a distal end of the delivery apparatus 200, such as to a nose cone 222 (as shown in FIG. 2), or to an alternate component at the distal end of the delivery apparatus 200 (e.g., a distal shoulder). An intermediate portion of the balloon 218 can overlay a mounting portion 224 of a distal end portion of the delivery apparatus 200 and a distal end portion of the balloon 218 can overlay a distal shoulder 226 of the delivery apparatus 200. The mounting portion 224 and the intermediate portion of the balloon 218 can be configured to receive a medical device (e.g., a prosthetic heart valve, a docking device, a prosthetic valve assembly, etc.) in a radially compressed state. For example, as shown schematically in FIG. 2, a medical device 250 (which can be one of the docking devices and/or one of the prosthetic heart valves described herein) can be mounted around the balloon 218, at the mounting portion 224 of the delivery apparatus 200.

The balloon shoulder assembly, including the distal shoulder 226, is configured to maintain the medical device 250 at a fixed position on the balloon 218 during delivery through the patient's vasculature.

In some examples, the delivery apparatus 200 can comprise more than one expansion mechanism (e.g., two balloons 218, etc.) at the mounting portion 224 that are independently expandable, such that more than one medical device can be mounted around the delivery apparatus 200 at a given time. For example, a docking device can be mounted around a first balloon 218 and a prosthetic heart valve can be mounted around a second balloon 218 of the delivery apparatus 200, such that one delivery apparatus 200 can implant both a docking device and a prosthetic heart valve.

The outer shaft 204 can include a distal tip portion 228 mounted on its distal end. The outer shaft 204 and the intermediate shaft 206 can be translated axially relative to one another to position the distal tip portion 228 adjacent to a proximal end of the mounting portion 224, when the medical device 150 is mounted in the radially compressed state on the mounting portion 224 (as shown in FIG. 2) and during delivery of the prosthetic valve to the target implantation site. As such, the distal tip portion 128 can be configured to resist movement of the medical device 250 relative to the balloon 218 proximally, in the axial direction, relative to the balloon 218, when the distal tip portion 228 is arranged adjacent to a proximal side of the mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the balloon 218. As such, fluid from the fluid source can flow to the fluid passageway from the annular space to inflate the balloon 218 and radially expand and deploy the medical device 250.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (e.g., for fine positioning at the implantation site). Further details on the delivery apparatus 200 can be found in PCT Application No. PCT/US2021/047056, which is incorporated by reference herein.

FIGS. 3 and 4 show an example of a prosthetic valve assembly 300 comprising the prosthetic heart valve 100 and a docking device 302. It should be noted that the prosthetic heart valve 100 is one example that can be used with the docking device 302. Any of various known prosthetic valves can be used with the docking device 302. The prosthetic heart valve can be any of various balloon-expandable heart valves, such as disclosed in U.S. Pat. No. 9,393,110, U.S. Publication No. 2018/0028310, U.S. Publication No. 2019/0365530, any of various self-expandable heart valves, such as disclosed in U.S. Pat. No. 9,155,619, or any of various mechanically expandable heart valves, such as disclosed in U.S. Publication No. 2018/0153689 or PCT Application No. PCT/US2021/052745, all of which prior patents and applications are incorporated herein by reference.

The prosthetic valve assembly 300 can be configured for implantation at an implant location within a native heart valve or a vessel. The docking device 302 (also referred to herein as a “docking structure”) has an inflow end 304 and an outflow end 306. The docking device 302 can comprise a base or main body 308 having an inner surface 310 that defines a lumen 312 extending between the inflow end 304 and the outflow end 306, such that the lumen 312 extends the entire length of the docking device 302. In some examples, the base 308 is generally cylindrical in shape. In other examples, the base 308 can comprise other shapes, for example, that correspond to a shape of the native heart valve or vessel at the implant location.

FIGS. 3 and 4 depict the prosthetic valve assembly 300 implanted in a heart 10 of a patient, in an expanded configuration. In particular, the prosthetic valve assembly 300 is positioned within the aortic annulus 12 and is seated against native leaflets 14. The docking device 302 can extend at least partially into the aortic root. As shown, the inflow end 304 of the docking device 302 is oriented towards the left ventricle 16 and the outflow end 306 is oriented towards the descending aorta 18. It should be appreciated that the prosthetic valve assembly 300 may be implanted within other native heart valves or within vessels of the patient.

The docking device 302 and the prosthetic heart valve 100 can each be radially expandable from a radially compressed state (e.g., during delivery) to a radially expanded state (e.g., expanded configuration shown in FIGS. 3-4). The base 308 of the docking device 302 can be made of any of various suitable plastically expandable materials (e.g., stainless steel, etc.) or self-expanding materials (e.g., Nitinol). For example, the base 308 can comprise a radially expandable annular metal frame and optionally can include one or more skirts or sealing layers covering the inner surface and/or the outer surface of the frame.

The base 308 can be non-porous to blood at least partially along the length of the base from the inflow end 304 to the outflow end 306 such that blood inside the lumen 312 cannot flow in a lateral direction (perpendicular to the flow of blood) through the main body. For example, the main body can be formed from a material that is non-porous to blood or can include an inner layer or outer layer formed from a material that is non-porous to blood (e.g., a fabric, a piece of tissue, or sheet of non-fabric material). In other examples, the main body can be formed with openings or is otherwise porous to blood at selected locations along its length to allow blood to flow laterally through the main body toward the coronary ostia.

When constructed of a plastically expandable material, the base 308 (and thus the docking device 302) can be crimped to a radially collapsed configuration on a delivery catheter or apparatus and then expanded inside a patient by an inflatable balloon or equivalent expansion mechanism. When constructed of a self-expandable material, the base 308 (and thus the docking device 302) can be crimped to a radially collapsed configuration and restrained in the collapsed configuration by insertion into a sheath or equivalent mechanism of a delivery catheter. Once inside the body, the docking device 302 can be advanced from the delivery sheath, which allows the docking device 302 to expand to its functional size.

In other instances, the base 308 of the docking device 302 or the entire docking device 302 can comprise an inflatable device (e.g., similar to a balloon) that receives an inflation media liquid to inflate. In such instances, the docking device can be delivered by a catheter in an uninflated state, positioned by the catheter at the desired implantation location, and then inflated with an inflation media. The inflation media can comprise a gas or a liquid (e.g., saline) or a flowable liquid (e.g., a biocompatible epoxy) that can harden in situ once injected into the docking device. Further details regarding inflatable implantable devices are disclosed in U.S. Publication No. 2006/0020333, which is incorporated herein by reference.

The prosthetic heart valve 100 can be positioned within the lumen 312 between the inflow end 304 and the outflow end 306 of the docking device 302. In particular, when the prosthetic valve assembly 300 is in the expanded configuration, as shown, an inner diameter of a central portion of the lumen 312 (e.g., the portion of the lumen 312 located between the inflow and outflow ends 304, 306 of the docking device 302, etc.) is larger than an outer diameter of the prosthetic heart valve 100, such that the prosthetic heart valve 100 can be positioned within the lumen 312 (e.g., within the central portion of the lumen 312, etc.).

In some examples, the prosthetic heart valve 100 can be positioned between the inflow end 304 and the outflow end 306 of the docking device 302 after the docking device 302 is positioned is implanted and/or deployed at an implant location (e.g., the native aortic annulus 12). In these examples, the docking device 302 can initially be introduced into a patient's vasculature using a first delivery apparatus (e.g., delivery apparatus 200 of FIG. 2) and deployed at the implant location. Then, the prosthetic heart valve 100 can introduced into the patient's vasculature using a second delivery apparatus (e.g., delivery apparatus 200 of FIG. 2) and the prosthetic heart valve 100 can be deployed within the lumen 312 of the docking device 302 (e.g., between the inflow end 304 and the outflow end 306).

Alternatively, the docking device 302 and the prosthetic heart valve 100 can both be introduced into the patient's vasculature using one delivery apparatus (e.g., a delivery apparatus comprising two balloons, etc.). In such an example, the docking device 302 can initially be deployed (expanded) at the implant location and then the delivery apparatus can be used to advance the prosthetic valve into the lumen 312 of the expanded docking device and expanded.

In other examples, the prosthetic heart valve 100 can be positioned within the lumen 312 between the inflow end 304 and the outflow end 306 of the docking device 302 prior to being introduced into the patient's vasculature. In these examples, the prosthetic valve assembly 300 (including the prosthetic heart valve 100 positioned within the docking device 302 with both components in a radially compressed state) is introduced into the patient's vasculature simultaneously using one expansion mechanism of a delivery apparatus (e.g., delivery apparatus 200 of FIG. 2) and then deployed at the implant location. In some instances, the docking device 302 can be self-expandable and the prosthetic heart valve 100 can be balloon-expandable. In this manner, when the prosthetic valve assembly 300 is deployed from a delivery apparatus, the docking device 302 can self-expand, and a balloon of the delivery apparatus can be used to expand the prosthetic heart valve 100 within the docking device 302. In these examples, the prosthetic valve assembly 300 can be deployed without requiring adjustment of the axial position of the prosthetic heart valve 100 relative to the docking device 302 prior to expanding the prosthetic heart valve 100.

When implanted, the prosthetic heart valve 100 can be configured to translate (e.g., move) longitudinally along a central longitudinal axis 318 of the prosthetic valve assembly 300 relative to the docking device 302. The longitudinal movement of the prosthetic heart valve 100 relative to the docking device 302 can be responsive to working cycles of the valve 100 (e.g., responsive to blood flow during systole and diastole, etc.). In particular, the prosthetic heart valve 100 can move longitudinally towards the outflow end 306 of the docking device 302 during forward flow of blood (FIG. 3) and the prosthetic heart valve 100 can move longitudinally towards the inflow end 304 of the docking device during the reverse flow of blood (FIG. 4).

In particular, FIG. 3 illustrates the prosthetic valve assembly 300 under the forward flow of blood (e.g., in a “forward flow configuration”). For example, when the prosthetic valve assembly 300 is positioned within the native aortic annulus 12 (as depicted in FIG. 3), the prosthetic valve assembly 300 is in the forward flow configuration during the systole phase of the flow cycle. In the forward flow configuration, the occluding structure 104 of the prosthetic heart valve 100 is in an open configuration, such that a first flow of blood F1 is permitted to flow through the prosthetic heart valve 100 in a first direction from the inflow end 110 to an outflow end 114 of the prosthetic heart valve 100. In the forward flow configuration, a second flow of blood F2 is also permitted to flow through the prosthetic valve assembly 300, between the docking device 302 and the prosthetic heart valve 100. In other words, the prosthetic valve assembly 300 includes a first blood flow pathway through the prosthetic heart valve 100 through which the first flow of blood F1 flows as well as a second blood flow pathway between an outer surface of the prosthetic heart valve 100 and the inner surface 310 of the docking device 302. As will be described in more detail below, the first flow F1 and the second flow F2 through the prosthetic valve assembly 300 can interact based on competitive flow principles to reduce pressure (e.g., pressure across the aortic annulus 12, etc.).

FIG. 4 illustrates the prosthetic valve assembly 300 under the reverse flow of blood (e.g., in a “reverse flow configuration”). For example, when the prosthetic valve assembly 300 is positioned within the native aortic annulus 12 (as depicted in FIG. 4), the prosthetic valve assembly 300 is in the reverse flow configuration during the diastole phase of the flow cycle. In the reverse flow configuration, the occluding structure 104 of the prosthetic heart valve 100 is in a closed configuration, such that blood (e.g., blood flowing in a second direction, opposite to the first direction, etc.) is prevented from flowing through the prosthetic heart valve 100. Blood is also prevented from flowing between the docking device 302 and the prosthetic heart valve 100 (e.g., due to the prosthetic heart valve 100 contacting and forming a seal with the inner surface 310 of the docking device 302, etc.). As such, in the reversed flow configuration, both the first and second blood flow pathways are closed and retrograde blood flow through the prosthetic valve assembly 300 is blocked.

The docking device 302 can include one or more stoppers configured to retain the prosthetic heart valve 100 between the outflow end 306 and the inflow end 304 of the docking device 302 during the working cycles of the prosthetic heart valve 100. In particular, the docking device 302 can include a first stopper 320 at the outflow end 306 configured to prevent the prosthetic heart valve 100 from translating longitudinally past or beyond the outflow end 306 of the docking device 302 during the forward flow of blood. In some examples, as depicted, the first stopper 320 comprises one or more inner radial projections. The inner radial projections 320 can define an opening 322 having an inner diameter that is smaller than an outer diameter of the prosthetic heart valve 100. In this manner, in the forward flow configuration (FIG. 3), the outflow end 114 of the prosthetic heart valve 100 can be translated longitudinally towards the outflow end 306 of the docking device 302 as blood flows in the first direction. In some examples, the outflow end 114 can translate longitudinally such that the outflow end 114 abuts the first stopper 320 and is prevented from translating farther longitudinally (e.g., past the outflow end 306 of the docking device 302, etc.).

The docking device 302 can also include a second stopper 324 at the inflow end 304 configured to prevent the prosthetic heart valve 100 from translating longitudinally past or beyond the inflow end 304 of the docking device 302 during the reverse flow of blood. In some examples, as depicted, the second stopper 324 can comprise a tapered or conical portion. For example, the lumen 312 can narrow from a first inner diameter at a central portion of the lumen 312 to a second, smaller inner diameter at the inflow end 304 of the docking device 302. In this way, the second stopper 324 can define an opening 326 having the second inner diameter which can be smaller than an outer diameter of the prosthetic heart valve 100. As such, in the reverse flow configuration (FIG. 4), the inflow end 110 of the prosthetic heart valve 100 can be translated longitudinally towards the inflow end 304 of the docking device 302 as blood flows in the second direction, until the inflow end 110 abuts the second stopper 324 and is prevented from translating farther longitudinally (e.g., past the inflow end 304 of the docking device 302, etc.).

In other examples, the first stopper 320 can comprise a conical or tapered portion (e.g., similar to the second stopper 324 illustrated in FIGS. 3-4, etc.) and the second stopper 324 can comprise one or more inner radial projections (e.g., similar to the first stopper 320 illustrated in FIGS. 3-4, etc.). It should be appreciated that the first stopper 320 and/or the second stopper 324 can comprise other structures configured to retain the prosthetic heart valve 100 between the outflow end 306 and the inflow end 304 of the docking device 302 during working cycles of the prosthetic heart valve 100.

In some examples, as depicted, the length of the docking device 302 along the central longitudinal axis 318 is longer than the length of the prosthetic heart valve 100 along the central longitudinal axis 318. In this manner, both ends of the prosthetic heart valve 100 are unable to abut the docking device 302 at any given time (e.g., only one end of the prosthetic heart valve 100 abuts a stopper of the docking device 302 at a time, neither end of the prosthetic heart valve 100 abuts the stoppers of the docking device 302, etc.). For example, during forward flow, the outflow end 114 of the prosthetic heart valve 100 can abut the first stopper 320 while the inflow end 110 does not abut the second stopper 324. As another example, during reverse flow, the inflow end 110 of the prosthetic heart valve 100 can abut the second stopper 324 while the outflow end 114 does not abut the first stopper 320. The difference in lengths of the docking device 302 and the prosthetic heart valve 100 enables the prosthetic heart valve 100 to translate longitudinally relative to the docking device 302.

As introduced above, the translation of the prosthetic heart valve 100 relative to the docking device 302 can facilitate pressure reduction. During forward flow, the first or primary flow of blood F1 is permitted to flow through the prosthetic heart valve 100 in the first direction (e.g., from an inflow end 110 to an outflow end 114 of the prosthetic heart valve 100, etc.). In addition to the first flow of blood F1, the secondary or competitive flow of blood F2 is permitted to flow through the prosthetic valve assembly 300 between the docking device 302 and the prosthetic heart valve 100 (e.g., as the prosthetic heart valve 100 translates from the inflow end 304 of the docking device 302 towards the outflow end 306 of the docking device 302, etc.). Stated another way, during forward flow, the high pressure serves to push the prosthetic heart valve 100 distally to the wider portion of the lumen 312, such that the main or primary stream F1 flows through the prosthetic heart valve 100, while the secondary stream F2 flows around the prosthetic heart valve 100, through the space formed between the prosthetic heart valve 100 and the wider portion of the lumen 312. The secondary stream F2 may cause competitive flow (e.g., directed back toward the aortic annulus, etc.) to form vortices that will disturb the main stream F1, thereby decreasing pressure gradient when the prosthetic heart valve 100 is open during the forward flow of blood. Advantageously, the secondary stream F2 can reduce pulsatile left ventricular afterload, which can be beneficial for treating hypertensive patients suffering from heart failure with preserved ejection fraction.

### Delivery Techniques

Described below are various delivery approaches for delivering a prosthetic valve assembly comprising a prosthetic valve and a docking device through a patient's vasculature via catherization. The term “prosthetic valve assembly” in this section is used to refer to a prosthetic valve, a docking device, or the combination of a prosthetic valve and docking device. In other words, for each described delivery approach, the prosthetic valve and the docking device can be delivered separately using respective delivery apparatuses, or alternatively the prosthetic valve and the docking device can be delivered through the patient's vasculature at the same time using the same delivery apparatus, as previously described.

For implanting a prosthetic valve assembly within the native aortic valve via a transfemoral delivery approach, the prosthetic valve assembly is mounted in a radially compressed state along the distal end portion of a delivery apparatus. The prosthetic valve assembly and the distal end portion of the delivery apparatus are inserted into a femoral artery and are advanced into and through the descending aorta, around the aortic arch, and through the ascending aorta. The prosthetic valve assembly is positioned within the native aortic valve and radially expanded (e.g., by inflating a balloon, actuating one or more actuators of the delivery apparatus, or deploying the prosthetic valve assembly from a sheath to allow the prosthetic valve to self-expand). Alternatively, a prosthetic valve assembly can be implanted within the native aortic valve in a transapical procedure, whereby the prosthetic valve assembly (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve assembly (on the distal end portion of the delivery apparatus) are introduced into the aorta through a surgical incision in the ascending aorta, such as through a partial J-sternotomy or right parasternal mini-thoracotomy, and then advanced through the ascending aorta toward the native aortic valve.

For implanting a prosthetic valve assembly within the native mitral valve via a transseptal delivery approach, the prosthetic valve assembly is mounted in a radially compressed state along the distal end portion of a delivery apparatus. The prosthetic valve assembly and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve assembly can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve assembly within the native tricuspid valve, the prosthetic valve is mounted in a radially compressed state along the distal end portion of a delivery apparatus. The prosthetic valve assembly and the distal end portion of the delivery apparatus are inserted into a femoral vein and are advanced into and through the inferior vena cava, and into the right atrium, and the prosthetic valve assembly is positioned within the native tricuspid valve. A similar approach can be used for implanting the prosthetic valve assembly within the native pulmonary valve or the pulmonary artery, except that the prosthetic valve assembly is advanced through the native tricuspid valve into the right. ventricle and toward the pulmonary valve/pulmonary artery.

Another delivery approach is a transatrial approach whereby a prosthetic valve assembly (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve assembly (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valve assemblies disclosed herein can be implanted using any of various delivery procedures and delivery devices known in the art.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Simulation

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Surgical Implementations

Any of the disclosed devices can be implanted within a patient's heart via conventional open-heart surgery. In such examples, the prosthetic valve and/or the docking device can be rigid structures, which need not be radially expandable from a compressed delivery state to an expanded, functional state. For example, the docking device can be a non-expandable structure and can be secured within a native heart valve (e.g., the native aortic valve) by suturing the docking device to the native annulus. Similarly, the prosthetic valve can be a non-expandable structure, such as a surgical prosthetic heart valve, such as disclosed in U.S. Publication No. 2020/0060812, which is incorporated herein by reference. Alternatively, any of the expandable prosthetic heart valves described above can be used in combination with a non-expandable docking device.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve assembly comprising: a docking structure having an inflow end and an outflow end, the docking structure having a lumen extending between the inflow end and the outflow end; and a prosthetic heart valve disposed within the lumen of the docking structure, the prosthetic heart valve comprising a frame and an occluding structure disposed within the frame, the occluding structure configured to regulate a flow of blood through the frame in one direction; wherein the docking structure is configured to allow the prosthetic heart valve to be movable relative to the docking structure in an axial direction between the inflow end and the outflow end of the docking structure during flow cycles of a heart.

Example 2. The prosthetic valve assembly of any example herein, particularly example 1, wherein the occluding structure is configured to permit a first flow of blood through the frame in a first direction and a second flow of blood between the frame and the docking structure in the first direction under the forward flow of blood.

Example 3. The prosthetic valve assembly of any example herein, particularly either example 1 or example 2, wherein the occluding structure is configured to prevent blood from flowing through the occluding structure under the reverse flow of blood.

Example 4. The prosthetic valve assembly of any example herein, particularly any one of examples 1-3, wherein an inner diameter of the lumen at the inflow end of the docking structure is smaller than an outer diameter of the prosthetic heart valve.

Example 5. The prosthetic valve assembly of any example herein, particularly example 4, wherein the lumen is tapered at the inflow end.

Example 6. The prosthetic valve assembly of any example herein, particularly any one of examples 1-5, wherein an inner diameter of the lumen at the outflow end of the docking structure is smaller than an outer diameter of the prosthetic heart valve.

Example 7. The prosthetic valve assembly of any example herein, particularly example 6, wherein the docking structure includes one or more inner radial projections that define the inner diameter of the lumen at the outflow end of the docking structure.

Example 8. The prosthetic valve assembly of any example herein, particularly example 6, wherein the lumen is tapered at the outflow end of the docking structure.

Example 9. The prosthetic valve assembly of any example herein, particularly any one of examples 1-8, wherein an inflow end of the prosthetic heart valve abuts an inflow end portion of the docking structure under the reverse flow of blood.

Example 10. The prosthetic valve assembly of any example herein, particularly example 9, wherein the inflow end of the prosthetic heart valve does not abut the inflow end portion of the docking structure under the forward flow of blood.

Example 11. The prosthetic valve assembly of any example herein, particularly any one of examples 1-10, wherein an outflow end of the prosthetic heart valve does not abut an outflow end portion of the docking structure under the reverse flow of blood.

Example 12. The prosthetic valve assembly of any example herein, particularly example 11, wherein the outflow end of the prosthetic heart valve abuts the outflow end portion of the docking structure under the forward flow of blood.

Example 13. The prosthetic valve assembly of any example herein, particularly any one of examples 1-12, wherein a length of the docking structure in the axial direction is longer than a length of the prosthetic heart valve in the axial direction.

Example 14. The prosthetic valve assembly of any example herein, particularly any one of examples 1-13, wherein the docking structure and the prosthetic heart valve are radially expandable.

Example 15. The prosthetic valve assembly of any example herein, particularly any one of examples 1-14, wherein the docking structure comprises a self-expandable material or a balloon-expandable material.

Example 16. A docking device comprising: a base having an inflow end and an outflow end, the base comprising a lumen extending from the inflow end to the outflow end, wherein the docking device is configured to receive a prosthetic heart valve such that the prosthetic heart valve is axially moveable within the lumen between the inflow end and the outflow end of the docking device during flow cycles of a heart.

Example 17. The docking device of any example herein, particularly example 16, wherein the lumen comprises a first inner diameter at the inflow end and a second inner diameter at a location between the inflow end and the outflow end; and wherein the first inner diameter is smaller than the second inner diameter.

Example 18. The docking device of any example herein, particularly example 17, wherein the lumen comprises a third inner diameter at the outflow end; and wherein the third inner diameter is smaller than the second inner diameter.

Example 19. The docking device of any example herein, particularly either example 17 or example 18, wherein the lumen is tapered from the second inner diameter to the first inner diameter.

Example 20. The docking device of any example herein, particularly any one of examples 17-19, wherein the lumen is tapered from the second inner diameter to the third inner diameter.

Example 21. The docking device of any example herein, particularly any one of examples 17-19, wherein the lumen is stepped from the second inner diameter to the third inner diameter.

Example 22. The docking device of any example herein, particularly any one of examples of 16-21, wherein the base is radially expandable from a compressed configuration to an expanded configuration.

Example 23. The docking device of any example herein, particularly example 22, wherein the base is formed from a self-expandable material or a balloon-expandable material.

Example 24. A prosthetic valve assembly comprising: a docking device having an inflow end and an outflow end; and a prosthetic heart valve positioned within the docking device between the inflow end and the outflow end; wherein the prosthetic valve assembly is configured to transition between a forward flow configuration and a reverse flow configuration in response to flow cycles of a heart, wherein the forward flow configuration comprises a first blood flow pathway extending through the prosthetic heart valve from the inflow end to the outflow end and a second blood flow pathway extending from the inflow end to the outflow end between an outer surface of the prosthetic heart valve and an inner surface of the docking device.

Example 25. The prosthetic valve assembly of any example herein, particularly example 24, wherein the first blood flow pathway and the second blood flow pathway are closed in the reverse flow configuration.

Example 26. The prosthetic valve assembly of any example herein, particularly either example 24 or example 25, wherein the inner surface of the docking device defines a lumen extending from the inflow end to the outflow end.

Example 27. The prosthetic valve assembly of any example herein, particularly example 26, wherein the prosthetic heart valve is positioned within the lumen.

Example 28. The prosthetic valve assembly of any example herein, particularly any one of examples 24-27, wherein the prosthetic heart valve is moveable in an axial direction relative to the docking device.

Example 29. The prosthetic valve assembly of any example herein, particularly example 28, wherein the prosthetic heart valve is in a first axial position relative to the docking device in the forward flow configuration and a second axial position relative to the docking device in the reverse flow configuration.

Example 30. The prosthetic valve assembly of any example herein, particularly example 29, wherein an outflow end of the prosthetic heart valve abuts the docking device in the first axial position.

Example 31. The prosthetic valve assembly of any example herein, particularly either example 29 or example 30, wherein an inflow end of the prosthetic heart valve does not abut the docking device in the first axial position.

Example 32. The prosthetic valve assembly of any example herein, particularly any one of examples 29-31, wherein an outflow end of the prosthetic heart valve does not abut the docking device in the second axial position.

Example 33. The prosthetic valve assembly of any example herein, particularly any one of examples 29-32, wherein an inflow end of the prosthetic heart valve abuts the docking device in the second axial position.

Example 34. The prosthetic valve assembly of any example herein, particularly any one of examples 24-33, wherein the inner surface is tapered at the inflow end.

Example 35. The prosthetic valve assembly of any example herein, particularly any one of examples 24-34, wherein the inner surface is tapered at the outflow end.

Example 36. A method of delivering a prosthetic valve assembly to an implantation location within a patient, the method comprising: advancing a docking device retained on a first delivery device to an implantation location within a patient; radially expanding the docking device at the implantation location; advancing a prosthetic heart valve retained on a second delivery device to the implantation location; positioning the prosthetic heart valve between inflow and outflow ends of the docking device; and radially expanding the prosthetic heart valve within the docking device.

Example 37. The method of any example herein, particularly example 36, wherein radially expanding the prosthetic heart valve comprises radially expanding the prosthetic heart valve to a working diameter that is less than an inner diameter of the docking device, thereby permitting axial movement of the prosthetic heart valve relative to the docking device.

Example 38. The method of any example herein, particularly either example 36 or example 37, wherein the prosthetic valve assembly comprises the prosthetic valve assembly of any example herein, particularly any one of examples 1-15.

Example 39. The method of any example herein, particularly either example 36 or example 37, wherein the docking device comprises the docking device of any example herein, particularly any one of examples 16-23.

Example 40. The method of any example herein, particularly either example 36 or example 37, wherein the prosthetic valve assembly comprises the prosthetic valve assembly of any example herein, particularly any one of examples 24-35.

Example 41. A method of delivering a prosthetic valve assembly to an implantation location within a patient, the method comprising: advancing a docking device retained on a first expansion mechanism of a delivery device and a prosthetic heart valve retained on a second expansion mechanism of the delivery device to an implantation location within a patient; radially expanding, by the first expansion mechanism, the docking device at the implantation location; positioning the prosthetic heart valve between inflow and outflow ends of the docking device; and radially expanding, by the second expansion mechanism, the prosthetic heart valve within the docking device.

Example 42. The method of any example herein, particularly example 41, wherein the prosthetic valve assembly comprises the prosthetic valve assembly of any example herein, particularly any one of examples 1-15.

Example 43. The method of any example herein, particularly example 41, wherein the docking device comprises the docking device of any example herein, particularly any one of examples 16-23.

Example 44. The method of any example herein, particularly example 41, wherein the prosthetic valve assembly comprises the prosthetic valve assembly of any example herein, particularly any one of examples 24-35.

Example 45. The method of any example herein, particularly any one of examples 41-44, wherein radially expanding the prosthetic heart valve comprises radially expanding, by the second expansion mechanism, the prosthetic heart valve to a working diameter that is less than an inner diameter of the docking device, thereby permitting axial movement of the prosthetic heart valve relative to the docking device.

Example 46. The method of any example herein, particularly any one of examples 41-45, wherein the first expansion mechanism is a balloon.

Example 47. The method of any example herein, particularly any one of examples 41-46, wherein the second expansion mechanism is a balloon.

Example 48. A method of delivering a prosthetic valve assembly to an implantation location within a patient, the method comprising: advancing a prosthetic valve assembly retained within a sheath of a delivery device to an implant location within a patient, the prosthetic valve assembly comprising a docking structure and a prosthetic heart valve positioned between an inflow end and an outflow end of the docking structure; deploying the prosthetic valve assembly from the sheath to allow the docking structure to self-expand at the implantation location; and radially expanding, by an expansion mechanism of the delivery device, the prosthetic heart valve within the docking device.

Example 49. The method of any example herein, particularly example 48, wherein the prosthetic valve assembly comprises the prosthetic valve assembly of any example herein, particularly any one of examples 1-15.

Example 50. The method of any example herein, particularly example 48, wherein the docking device comprises the docking device of any example herein, particularly any one of examples 16-23.

Example 51. The method of any example herein, particularly example 48, wherein the prosthetic valve assembly comprises the prosthetic valve assembly of any example herein, particularly any one of examples 24-35.

Example 52. The method of any example herein, particularly any one of examples 48-51, wherein radially expanding the prosthetic heart valve comprises radially expanding, by the expansion mechanism, the prosthetic heart valve to a working diameter that is less than an inner diameter of the docking device, thereby permitting axial movement of the prosthetic heart valve relative to the docking device.

Example 53. The method of any example herein, particularly any one of examples 48-52, wherein the expansion mechanism is a balloon.

Example 54. A prosthetic valve assembly comprising: a docking device having an inflow end and an outflow end and first and second stops at axially spaced locations; and a prosthetic heart valve configured to be positioned within the docking device between the first and second stops; wherein the docking device is configured to allow the prosthetic heart valve to move axially within the docking device in first and second directions between the first and second stops during flow cycles of a heart.

Example 55. The prosthetic valve assembly of any example herein, particularly example 54, wherein the prosthetic valve assembly is configured to transition between a forward flow configuration and a reverse flow configuration in response to flow cycles of a heart, wherein the forward flow configuration comprises a first blood flow pathway extending through the prosthetic heart valve from the inflow end to the outflow end and a second blood flow pathway extending from the inflow end to the outflow end between an outer surface of the prosthetic heart valve and an inner surface of the docking device.

Example 56. The prosthetic valve assembly of any example herein, particularly any one of examples 54-55, wherein the docking device comprises an annular main body having an inner surface defining a lumen.

Example 57. The prosthetic valve assembly of any example herein, particularly example 56, wherein the first stop extends radially inwardly into the lumen.

Example 58. The prosthetic valve assembly of any example herein, particularly example 57, wherein the first stop comprises a plurality of inwardly extending projections.

Example 59. The prosthetic valve assembly of any example herein, particularly any one of examples 56-58, wherein the second stop comprises a tapered portion of the inner surface.

Example 60. The prosthetic valve assembly of any example herein, particularly any one of examples 56-59, wherein the main body is non-porous to blood at least partially along a length of the main body from the inflow end to the outflow end.

Example 61. A prosthetic valve assembly of any example herein, particularly, any of examples 1-15, 24-35, and 54-60, wherein the prosthetic valve assembly is sterilized.

Example 62. A docking device of any example herein, particularly, any of examples 16-23, wherein the docking device is sterilized.

The features described herein with regard to any example can be combined with other features described in any one or more of the other examples, unless otherwise stated. For example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A prosthetic valve assembly comprising:
a docking structure having an inflow end and an outflow end, the docking structure having a lumen extending between the inflow end and the outflow end; and
a prosthetic heart valve disposed within the lumen of the docking structure, the prosthetic heart valve comprising a frame and an occluding structure disposed within the frame, the occluding structure configured to regulate a flow of blood through the frame in one direction;
wherein the docking structure is configured to allow the prosthetic heart valve to be movable relative to the docking structure in an axial direction between the inflow end and the outflow end of the docking structure during flow cycles of a heart.

2. The prosthetic valve assembly of claim 1, wherein the occluding structure is configured to permit a first flow of blood through the frame in a first direction and a second flow of blood between the frame and the docking structure in the first direction under the forward flow of blood.

3. The prosthetic valve assembly of claim 1, wherein the occluding structure is configured to prevent blood from flowing through the occluding structure under the reverse flow of blood.

4. The prosthetic valve assembly of claim 1, wherein an inner diameter of the lumen at the inflow end of the docking structure is smaller than an outer diameter of the prosthetic heart valve.

5. The prosthetic valve assembly of claim 4, wherein the lumen is tapered at the inflow end.

6. The prosthetic valve assembly of claim 1, wherein an inner diameter of the lumen at the outflow end of the docking structure is smaller than an outer diameter of the prosthetic heart valve.

7. The prosthetic valve assembly of claim 6, wherein the docking structure includes one or more inner radial projections that define the inner diameter of the lumen at the outflow end of the docking structure.

8. The prosthetic valve assembly of claim 6, wherein the lumen is tapered at the outflow end of the docking structure.

9. The prosthetic valve assembly of claim 1, wherein a length of the docking structure in the axial direction is longer than a length of the prosthetic heart valve in the axial direction.

10. A prosthetic valve assembly comprising:
a docking device having an inflow end and an outflow end and first and second stops at axially spaced locations; and
a prosthetic heart valve configured to be positioned within the docking device between the first and second stops;
wherein the docking device is configured to allow the prosthetic heart valve to move axially within the docking device in first and second directions between the first and second stops during flow cycles of a heart.

11. The prosthetic valve assembly of claim 10, wherein the prosthetic valve assembly is configured to transition between a forward flow configuration and a reverse flow configuration in response to flow cycles of a heart, wherein the forward flow configuration comprises a first blood flow pathway extending through the prosthetic heart valve from the inflow end to the outflow end and a second blood flow pathway extending from the inflow end to the outflow end between an outer surface of the prosthetic heart valve and an inner surface of the docking device.

12. The prosthetic valve assembly of claim 10, wherein the docking device comprises an annular main body having an inner surface defining a lumen.

13. The prosthetic valve assembly of claim 12, wherein the first stop extends radially inwardly into the lumen.

14. The prosthetic valve assembly of claim 13, wherein the first stop comprises a plurality of inwardly extending projections.

15. The prosthetic valve assembly of claim 12, wherein the second stop comprises a tapered portion of the inner surface.

16. The prosthetic valve assembly of claim 12, wherein the main body is non-porous to blood at least partially along a length of the main body from the inflow end to the outflow end.

17. A docking device comprising:
a base having an inflow end and an outflow end, the base comprising a lumen extending from the inflow end to the outflow end, wherein the docking device is configured to receive a prosthetic heart valve such that the prosthetic heart valve is axially moveable within the lumen between the inflow end and the outflow end of the docking device during flow cycles of a heart.

18. The docking device of claim 17, wherein the lumen comprises a first inner diameter at the inflow end and a second inner diameter at a location between the inflow end and the outflow end; and
wherein the first inner diameter is smaller than the second inner diameter.

19. The docking device of claim 18, wherein the lumen comprises a third inner diameter at the outflow end; and
wherein the third inner diameter is smaller than the second inner diameter.

20. A method of delivering a prosthetic valve assembly to an implantation location within a patient, the method comprising:
advancing a docking device retained on a first expansion mechanism of a delivery device and a prosthetic heart valve retained on a second expansion mechanism of the delivery device to an implantation location within a patient;
radially expanding, by the first expansion mechanism, the docking device at the implantation location;
positioning the prosthetic heart valve between inflow and outflow ends of the docking device; and
radially expanding, by the second expansion mechanism, the prosthetic heart valve within the docking device.

21. The method of claim 20, wherein radially expanding the prosthetic heart valve comprises radially expanding, by the second expansion mechanism, the prosthetic heart valve to a working diameter that is less than an inner diameter of the docking device, thereby permitting axial movement of the prosthetic heart valve relative to the docking device.
